(19) United States
(12) Patent Application Publication
(10) Pub. No.: US 2012/0018313 A1
(43) Pub. Date: Jan. 26, 2012

(54) NOVEL BIOTRACERS AND USES THEREOF FOR CONTROLLING FILTRATION PLANTS

(75) Inventors: Christelle Guigui, Toulouse (FR); Corinne Cabassud, Saint-Orens de Gameville (FR); Sandrine Alfenore, Castanet -Tolosan (FR); Stéphane Mathe, Toulouse (FR); Laurence Soussan, Graulhet Cedex (FR)

(73) Assignee: CENTRE NATIONAL DE LA RECHERCHE SCIENTIFIQUE (C.N. R.S), Paris (FR)

(21) Appl. No.: 13/141,262

(22) PCT Filed: Dec. 16, 2009
(86) PCT No.: PCT/FR2009/052571

§ 371 Date: Oct. 7, 2011

(30) Foreign Application Priority Data
Dec. 22, 2008 (FR) 08 58938

(51) Int. Cl. C12Q 1/02; C12N 7/02; C12N 7/00

(52) U.S. Cl. 205/777.5; 435/235.1; 435/239; 204/403.01

(57) ABSTRACT

The present invention relates to novel biotracers, to a method for preparing same, and to a method for detecting the biotracers and for monitoring filtration systems.

Membrane filtering methods are used in numerous fields to concentrate or purify liquids. In some applications (water treatment, agri-foodstuff industry . . . ) the ability to monitor the condition (the term integrity is also used) of membranes when in use is of importance, in order to ensure the quality of the end product.

As an example, mention may be made of water supply and treatment plants equipped with filtration systems to permit limiting of the content of biocontaminants and other compounds (viruses, bacteria, etc.) in the water produced. Membrane technology (in particular ultra-filtration) is widely used on account of its extensive virus removal capability.

The removal (or retaining) rate of a filtration system is defined by the percentage of retained compounds.

To guarantee the monitoring and quality of treatment plants it is necessary to be able to characterize retention by the filtration systems used, both in-line (directly on the installations) and dynamically (as a function of time), in order to achieve objective qualification of the retaining performance level of the filtering systems, to increase the guaranteed health safety level of plants and to reduce the demand for chemical disinfectants.

Membrane cut-off thresholds (i.e. 90% retaining of the target compound) are currently assessed by means of very small tracers of polymer or protein type such as PEGs (polyethylene glycol) and dextrans. These tracers have properties different to those of the viruses, whether in terms of size, shape or density. They are therefore not adapted for mimicking the behaviour of viruses at the time of filtering, and therefore cannot satisfactorily characterize the retaining dynamics of membrane systems when filtering. It is therefore necessary to provide a tracer which is easily detectable and quantifiable in-line, and which is capable of best mimicking the filtering behaviour of viruses.

Bacteriophages (also called phages) are viruses of bacteria non-pathogenic for man and his environment. They are frequently used as reference microorganisms to quantify the virus retention of membrane systems (Membrane Filtration Guidance Manual: Environmental Protection Agency, 2005; U.S. Pat. No. 5,645,984 A, and U.S. Pat. No. 5,731,164 A). They are indeed very similar to pathogenic viruses conveyed by water, from the viewpoint of size, shape and surface properties. However, existing methods to quantify bacteriophages (lysis plaque count, quantitative PCR, flow cytometry . . . ) are not sufficiently rapid and representative of filtered volumes for the targeted applications.

Substitutes for pathogenic viruses have been envisaged in the literature. Particular mention may be made of gold nanoparticles detected by potentiometry (Gitis et al, Journal of Membrane Science, 276, 2006, 1999-207). However, despite similar sizes, these particles are not representative of viruses since these nanoparticles have much greater density and a distinctly smoother surface than viruses. In addition, they are much less deformable.

Under another approach, tracers such as bacteria are known whose surface has been modified with paramagnetic particles so that it is possible to collect and detect said bacteria through the application of a magnetic field (US 2003/168408). However, the implementing of detection by said tracers remains difficult. In another study, modified bacteriophages have also been envisaged as tracers (Gitis et al., Water Research, 36 (2002), 4227-4234 and application WO2007/046095). One of the proposed modifications concerns the grafting of fluorochromes on the surface of MS2 bacteriophages so that it is possible directly to detect said modified bacteriophages by fluorometry. This pertinent approach remains limited however through the small size of analyzable volumes (maximum 1 mL, continuous feeding of the fluorometric cell being impossible due to the strong fouling nature of all biological suspensions). The grafting of enzymes (having a molecular weight more than 200 times higher than the fluorochromes) on the surface of T4 bacteriophages has also been envisaged. In this particular case, the limitations are chiefly due to the large size of the T4 phage (measuring around 150 nm in length and 78 nm in width) thereby making use of the tracer obsolete in ultrafiltration or nanofiltration membranes and in the associated detection method (ECL chemiluminescence) in which small volumes are analyzed (of the order of 20 microlitres). It will be noted that for said tracer, the authors did not provide any characterization of the described tracer, and there is no means for quantifying the number of tracers present in a sample and hence for providing detection threshold values.

It is therefore desirable for biotracers to be provided that are as representative as possible of viruses, allowing rapid, continuous detection compatible with large volumes. Also, it is desirable to be able to ensure the reproducible, quantitative characterization of said tracer.

The present invention therefore concerns a novel detection method by amperometry for detecting biotracers consisting of a bacteriophage labelled on its surface by one or more enzymatic probes grafted via one or more molecules of activated biotin previously attached to one or more proteins of the capsid of said bacteriophage.

Therefore the method of the invention for detecting biotracers in a sample to be analyzed preferably comprises:
preparing an amperometric cell comprising:
   a working electrode,
   a counter-electrode,
   a reference electrode,
these three electrodes being connected via a potentiostat-galvanostat;
   a solution comprising the sample to be analysed and at least one biotracer as defined in the foregoing,
   an electrolyte,
   an oxidant,
   an electron donor R,
      and
measuring the current thus generated by amperometry.

Preferably the electrolyte comprises a buffer to fix the pH of the solution.

Preferably, the detection method of the invention is such that the lower detection threshold of the biotracers is generally equal to or more than 10², more preferably equal to or more than 10⁴ pfu/mL. There is no upper detection limit of biotracer concentration and, if needed, dilution of the sample can be performed before analysis.

As phages suitable for the invention, any type having proteins on the surface thereof is suitable.

The phage used is preferably chosen from among phages which can easily be amplified in bacterial strains. Preferably, the chosen phages have replication cycles in non-pathogenic bacteria. These phages can be chosen from any known family of phages. The type of phage (size) is particularly chosen in relation to the type of filtration to be characterized (in particular with respect to the cut-off threshold of the membrane). For filtration of microfiltration (MF), ultrafiltration (UF) and/or nanofiltration (NF) type, the phage used is most preferably the MS2 phage. However, phages of greater size or molecular weight may also be suitable for the microfiltration method.

Therefore, the MS2 phages in particular are spherical viruses of mean diameter 30 nm, surrounded by a protein shell called a capsid, and are suitable for the invention. The bacteriophages can be obtained from approved bodies such as ATCC or Institut Pasteur. The MS2 bacteriophage for example corresponds to the ATCC strain 15 597-B1.

The phages thus labelled are also called biotracers.

The terms phages and bacteriophages are interchangeable and relate to viruses which infect bacteria.

A biotin molecule is attached to one or more proteins of the phage capsid. The biotin used is modified so as to offer a reactive terminal function. Said modified biotin is called an activated biotin herein. Biotin is an extremely small, hydrophilic molecule enabling it to diffuse easily towards its attachment sites to act thereat. It is widely used for biochemical tests for its ability to form covalent bonds with proteins and on account of its small size. A spacer can also be inserted before the reactive terminal function of the activated biotin.

The biotin molecules used are activated to react with priority sites of the protein capsid of the phage used. For example, biotin molecules activated to react with the —NH₂ primary amine groups particularly present in lysine amino acids, were chosen for the MS2 phage so as to set up a covalent bond of amide type. The capsid of the phages may effectively be composed of proteins containing at least one lysine. This is particularly the case with MS2 phages whose capsid is composed of 180 identical proteins each having an accessible lysine site for this type of grafting (Lin et al., J. Mol. Biol., 1967, 25(3), 455-463).

As an illustration, the activated biotin molecules can be commercially obtained from Perbio Science for example, e.g. EZ-Link Sulfo-NHS-LC-Biotin.

The enzymatic probes are attached to the activated biotin via very strong interaction (characterized by a high association constant) similar to a covalent bond over a wide pH and temperature range.

As enzymatic probes, any complex can be used that is composed of:
a protein carrier able to interact with the activated biotin, and
an enzyme of oxidoreductase type.

The choice of enzymatic probe (enzyme/protein carrier complex) is generally dependent upon:
the final size of the desired biotracer with respect to the membrane to be characterized,
enzyme-detection sensitivity,
and the interactions of said molecules with the membrane of the filtering system to be characterized.

Said protein carriers of the enzymatic probe can be chosen for example from among neutravidin, avidin or streptavidin.

As enzyme, particular mention may be made of the oxidoreductase Horse Radish Peroxidase (HRP), commonly used in immunology for its strong activity and low molecular weight.

Therefore, as enzymatic probes, preference is particularly given to the neutravidin-HRP complex, marketed by Perbio Science for example.

Preferably the biotracers of the invention are such that:
they are available in a suspension in which there are no free probes, and/or
the mean number of enzymatic probes grafted per phage ranges from 20 to 150, and is preferably between 40 and 60 in relation to the conditions of synthesis, and/or
the mean catalytic activity of the biotracers, k_{cat}, lies between 2·10⁴ and 4·10⁴ min⁻¹.

When implementing the method of the invention, an oxidation reaction of the electron donor R occurs in the presence of the oxidant and the biotracer of the invention, being oxidized to the corresponding oxidized donor O, said oxidation reaction being catalyzed by said biotracer. More precisely, the oxidation reaction is catalyzed irreversibly by the enzyme of the enzymatic probe of the biotracer.

The oxidized donor O thus formed diffuses towards the working electrode. A polarisation voltage <<Vₚₒₗₐᵣᵢₛₐₜᵢₒₙ>>, lower than the equilibrium potential of the oxido-reducing pair, oxidized donor O/electron donor R, is applied between the working electrode and the reference electrode, so that the oxidized donor O which has arrived in the vicinity of the working electrode, is reduced to an electron donor compound R thereby generating a reduction current I (called cathodic current). The reduction current I, measured over time is directly proportional to the concentration of oxidized donor O formed in solution, hence to the total quantity of grafted enzymatic probes and hence to the total quantity of grafted enzymes.

Said detection method can therefore be qualitative or quantitative.

As electron donor R, particular mention may be made of iodide or 3,3′,5,5′-tetramethylbenzidine (commonly called TMB). As oxidant, a suitable oxidant is one allowing the oxido-reduction reaction catalysed by the enzyme of the enzymatic probe grafted on the biotracer. For example, hydrogen peroxide is advantageously suitable for the HRP enzyme. The HRP enzyme effectively catalyses the oxidation reaction of R irreversibly, in the presence of hydrogen peroxide, so as to form the corresponding oxidized donor O and water, as per the reaction:

Electron donor (R)+H₂O₂→Oxidized electron donor (O)+H₂O

The ratio of oxidant concentration to the concentration of electron donor (R) may vary from 1 to 10. This choice particularly contributes towards limiting the spontaneous oxidation reaction of the electron donor R in the presence of the oxidant.

The electrolytes can be chosen from among any electrolyte usually used, such as NaCl, KCl. The concentration of electrolyte is generally less than 1 M, preferably less than 0.3 M. The pH of the solution to be analysed is chosen so as best to promote the activity of the grafted enzyme and least to promote the spontaneous oxidation of the electron donor R. If the enzyme is HRP and the electron donor is TMB, the pH can be fixed at between 5 and 7.

The buffers suitable for the invention can be chosen from among any buffer usually used, such as citrate-phosphate, phosphate, etc.

The working electrode is advantageously a rotating disk electrode (RDE) which may be in particular consist of platinum, glassy carbon or gold. It is to be noted that the sizing of the active surface of the working electrode depends upon the quantity of oxidized donor O present in solution, and hence upon the volume of the solution to be analysed: the larger the volume of solution, the larger the active surface which can be chosen.

The counter-electrode is advantageously a platinum electrode. In general, the surface of the working electrode is a small surface compared with the surface of the counter-electrode. Therefore, by way of illustration, the surface of the platinum counter-electrode may be 5 to 10 times larger than the surface of the working electrode. The reference electrode can be chosen from among the reference electrodes usually used in electrochemistry (such as Ag/AgCl).

The polarisation voltage is generally such that the spontaneous oxidation of the electron donor R is the most disadvantaged and the reduction of the oxidized donor O at the work electrode is the best advantaged.

Any potentiostat-galvanostat assembly operating in potentiostat or galvanostat mode can be used; however, preference is given to the galvanostat mode allowing current measurement. The analysis time is generally ranges from 1 to 30 minutes.

The present invention also concerns the method for monitoring a filtration system.

Said method comprises:
adding at least one biotracer consisting of a labelled bacteriophage comprising on its surface one or more enzymatic probes grafted via one or more molecules of activated biotin previously attached to one or more proteins of the capsid of said bacteriophage, to the feed of said system,
the step for detecting biotracers in the permeate (or retentate) of said filtration system according to the invention.

Preferably, the step for detecting biotracers in the permeate (or retentate) is performed in a sample of said permeate.

The detection of current in the sample to be analyzed (for example a permeate sample) allows identification of the presence of the biotracers mimicking viruses in the discharged water.

If it is desired to have quantification of the biotracers in the permeate (or retentate), this can be particularly obtained with the step comprising the comparison of measured current with reference values.

In addition, the obtaining of reference values by calibrating the current in relation to the quantity (or concentration) of tracers provides access, merely via current measurement, to the quantity (or concentration) of biotracers in the analysed sample.

According to one particular embodiment, said monitoring method allows determination of the percent removal by said filtration system. Percent removal Ab is defined as the decimal logarithm of the ratio between the feed concentration of biotracers Ca and the concentration of biotracers in the permeate Cp:

Ab=log(Ca/Cp) [equation 0]

For this purpose, the said method further comprises:
the step for detecting biotracers in the feed of said system, then
the step for detecting biotracers in the permeate and/or retentate of said system, then
comparison of the current in the permeate (or retentate) with the current obtained in the feed.

Preferably, the procedure entails sampling of the feed and permeate (or retentate), but analysis could be conducted in-line using a conventional flow amperometric cell.

A further subject of the invention concerns the use of conventional amperometric cells for detecting enzymatic activities.

The biotracers consists of a bacteriophage of labelled MS2 type, such that:
the said bacteriophage has proteins on the surface of its capsid,
a molecule of activated biotin is grafted on one or more of said proteins, and
one or more enzymatic probes are attached to said biotin.

A further subject of the invention therefore concerns a biotracer consisting of a labelled bacteriophage, such that said bacteriophage is a MS2 phage which comprises on its surface one or more enzymatic probes grafted via one or more molecules of activated biotin previously attached to one or more proteins of the capsid of said bacteriophage.

Preferably, the biotracer according to the invention may comprise the embodiments described in the foregoing with reference to the method of the invention.

According to a further subject of the present invention, it also concerns the method for preparing said biotracers.

Therefore, the said method comprises the following steps:
immobilizing a molecule of activated biotin on one or more proteins present on the surface of the bacteriophage to be labelled, then
grafting one or more enzymatic probes on said biotin(s).

According to one particular aspect, the method of the invention further comprises the step for quantifying the mean number of grafted probes per bacteriophage. The method of the invention then allows the characterization of said biotracers.

The techniques for immobilizing biotin and for attaching the enzymatic probe are well known to those skilled in the art. Preferably, said molecules of activated biotin are immobilized on the lysines of the surface proteins of the bacteriophage.

According to one preferred embodiment, excess biotin is used so that a molecule of activated biotin is immobilized on each accessible lysine of the proteins on the surface of the bacteriophage to be labelled.

The method may also comprise the subsequent step of purification, preferably by size exclusion high performance liquid chromatography (also called HPLC-SEC), to separate, collect and assay the free enzymatic probes. The mass of grafted probes can be deduced from the difference between the known mass of the enzymatic probes injected into the chromatography column and the mass of free enzymatic probes collected at the exit of the column. Knowledge of the mass of grafted enzymatic probes can therefore by used to assess the mean number of enzymatic probes grafted per phage. Said quantification cannot be conducted if purification is performed by dialysis in particular, since the probes are too much diluted in the dialysis waters to allow assay thereof.

If necessary, a purification step can be conducted after immobilization of the activated biotin on the phages, the phages then being separated from the non-grafted molecules of activated biotin. This purification step is advantageously performed using HPLC-SEC.

The method, in one preferred embodiment, may comprise a preliminary step for producing phages to be labelled. This production step comprises the steps consisting of:
amplifying the phages to be labelled;
placing the phages thus amplified in suspension;
purifying the phages.

The phages are amplified in the presence of host bacteria, for example following the solid phase protocol recommended by the ATCC. It will be noted that this amplification can be conducted in a liquid or solid medium. The amplification time and conditions will depend upon the type of phage used and can easily be determined by persons skilled in the art.

The phages thus amplified are then replaced in suspension in a liquid medium, for example in saline water or preferably in neutral phosphate buffered saline (also commonly called PBS) to be purified.

The purification step may comprise lysis of the bacteria, for example with chloroform, and one or more centrifuging steps followed by filtrations. The phages thus purified can be stored in a liquid medium, preferably in PBS.

The method may also comprise count determinations of the phages thus amplified and purified, for example using HPLC-SEC or phage count techniques in an aqueous medium (in particular as per standard ISO 10705-1).

A further subject of the invention also concerns a kit comprising:
a solution comprising at least one biotracer composed of a labelled bacteriophage, comprising on its surface one or more enzymatic probes grafted via one or more molecules of activated biotin previously attached to one or more proteins of the capsid of said bacteriophage; and
an amperometric cell comprising a working electrode, a counter-electrode and a reference electrode,
an electrolyte,
an electron donor R and,
an oxidant.

The electron donors, the oxidants, the electrolytes, the buffers, the working electrode, the counter-electrode and the reference electrode can be chosen from among those preferred for the biotracer detection method of the invention.

The solution may contain a biotracer concentration of up to 10¹² pfu/mL.

Preferably, the electrolyte comprises a buffer.

The invention will be better understood with the help of the following figures:

FIG. 1 schematically illustrates a biotracer according to the invention, in which reference 1 denotes the protein of the capsid of the phage, 2 denotes the molecule of activated biotin, 4 denotes the protein carrier of the enzymatic probe (for example the neutravidin molecule) covalently bound to one or two molecules of HRP C enzymes denoted 5, the enzymatic probe being denoted 3.

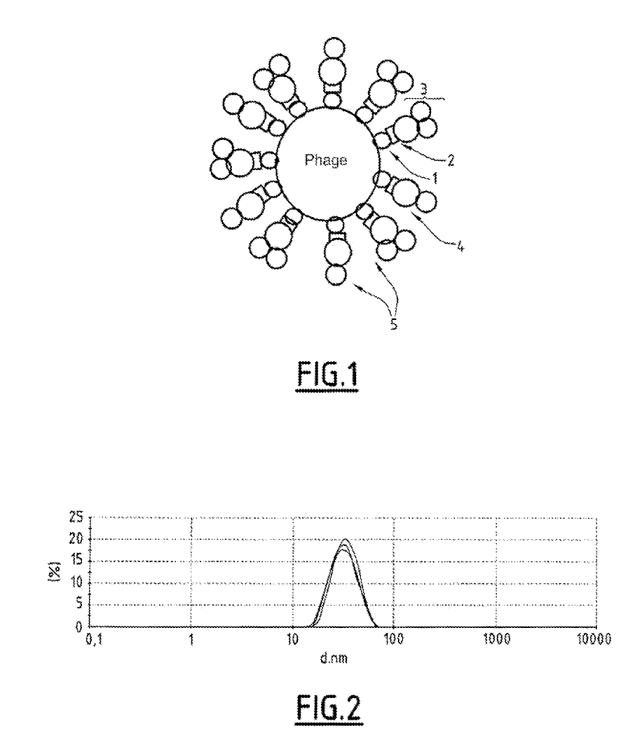

FIG. 2 shows the size distribution of a batch of phages obtained in Example 1 (after amplification and extraction with chloroform).

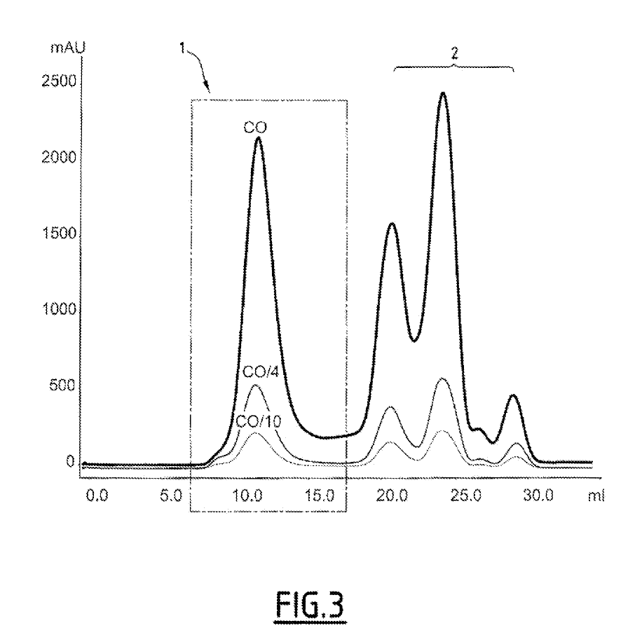

FIG. 3 gives the chromatograms at 254 nm of suspensions of native phages amplified and extracted with chloroform at concentrations C0, C0/4 and C0/10, in which 1 represents the peak of the native phages and 2 the protein domain.

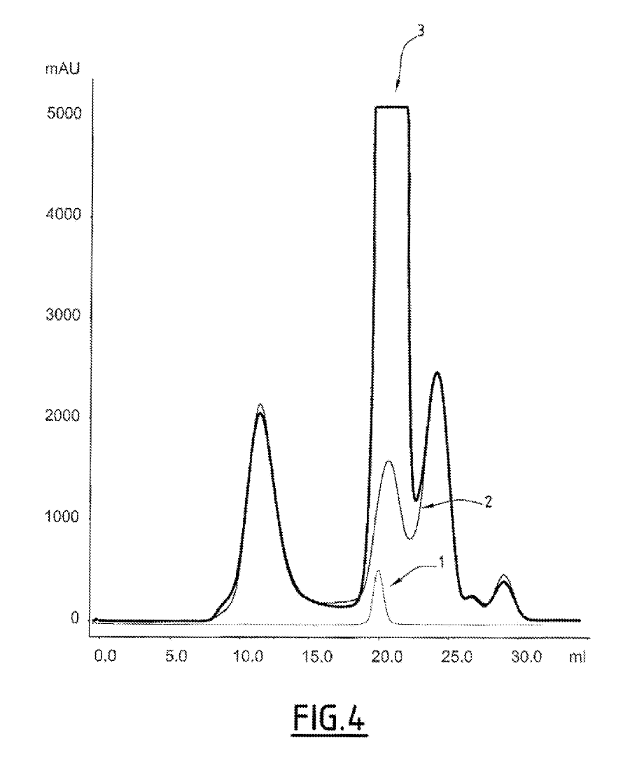

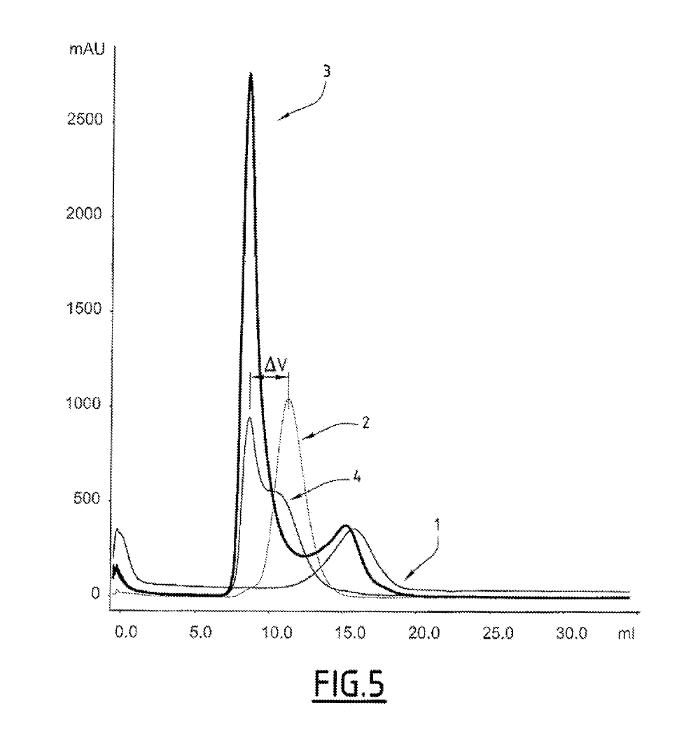

FIG. 4 gives the chromatograms obtained at 254 nm of biotin alone (curve 1), of native phages (curve 2), of biotin-labelled phages (curve 3).

FIG. 5 gives the chromatograms at 210 nm of the enzymatic probe alone (curve 1), of a purified suspension of biotin-labelled phages (curve 2), of a mixture of biotracers and probes in excess (curve 3), of a mixture of biotracers and biotin-labelled phages in cases when the probes are in shortage (curve 4).

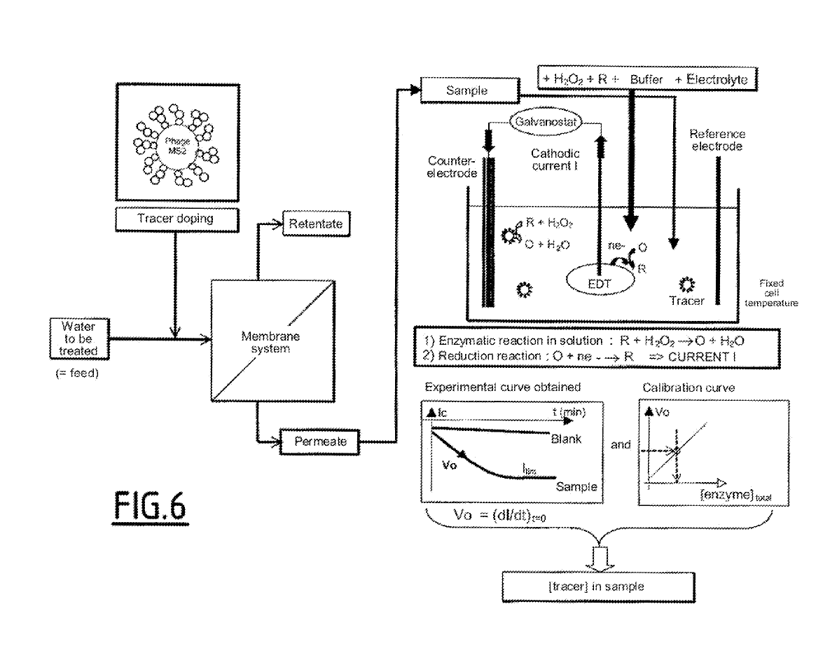

FIG. 6 gives a schematic description of a detection method in which, by way of illustration, a MS2 phage is used and it is the permeate that is analyzed. Alternatively, the retentate or feed could also be analyzed, and another phage could be used.

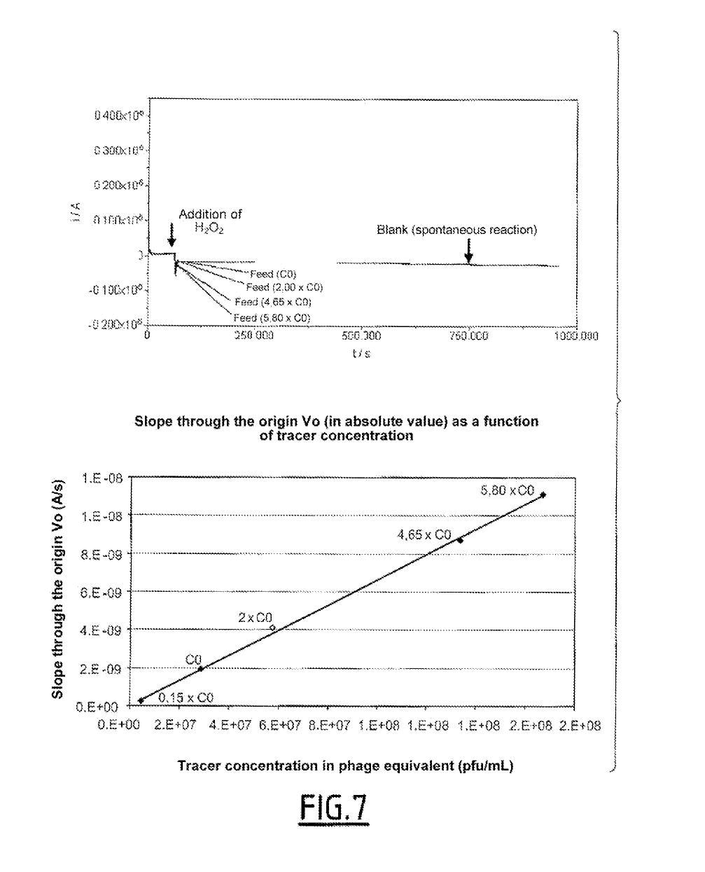

FIG. 7a gives an example of amperometric curves of one same tracer feed (4 samples of increasing volume); FIG. 7b shows the calibration line associated with the example (giving the slopes measured as a function of tracer concentrations in the measuring cell, expressed in native phage count units).

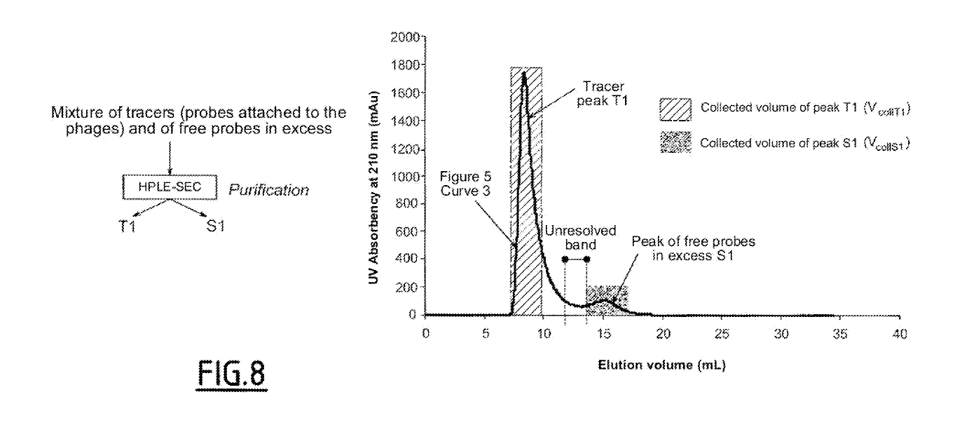

FIG. 8 is a chromatogram at 210 nm of a mixture of tracers and probes in excess (curve 3 in FIG. 5), showing the peaks of the tracers and probes in excess T1 and S1 respectively, and the collected volumes of each peak.

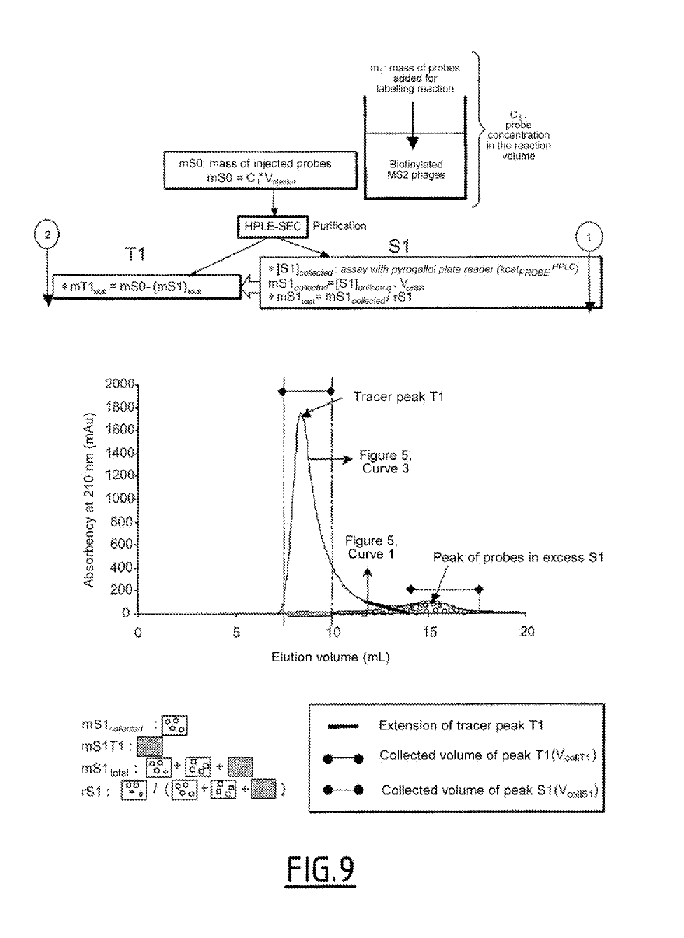

FIG. 9 illustrates details of the methodology applied to access the mass of probes grafted on the phages.

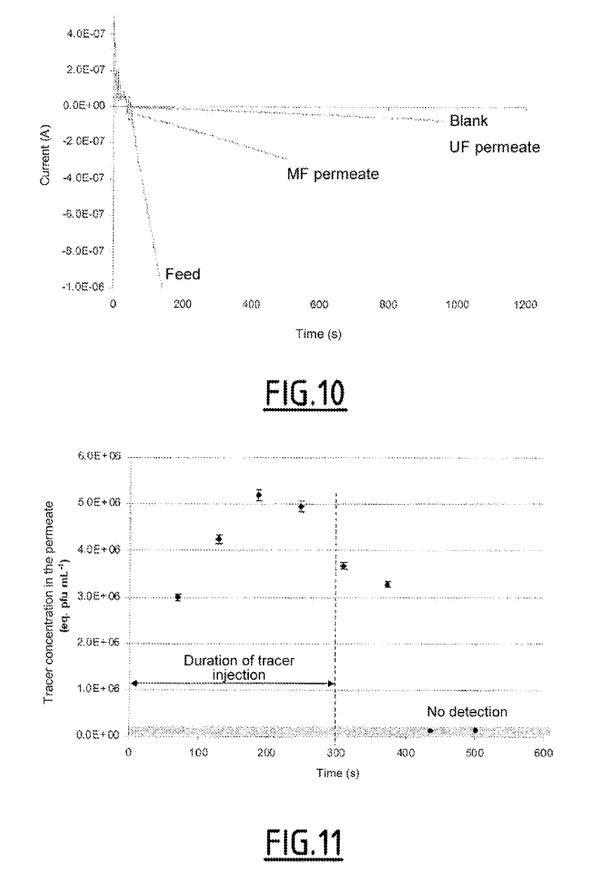

FIG. 10 shows an example of amperometric responses obtained by analysing the feed and permeates collected at the end of filtration, for a microfiltration (MF) and ultrafiltration (UF) membrane.

FIG. 11 gives an example of dynamic characterization of retention obtained with a damaged module of ultrafiltration hollow fibres used for producing potable water. This module consisting of 15 fibres was deliberately compromised by forming a circular defect of 25 μm using laser on one of the 15 fibres. During this experiment, tracer increments were applied in particular for around one third of the total filtration time (conducted frontally at constant transmembrane pressure), and the permeate was collected during filtration for analysis. The tracer concentrations measured in the permeate collected during filtration, are given as a function of filtering time.

The following examples are given by way of illustration of the present invention and are non-limiting.

### EXAMPLES

### Example 1

### Preparation of the Biotracer

A biotracer is illustrated in FIG. 1.

Reagents:
MS2 bacteriophages (ATCC, strain 15 597-B1),
Non-pathogenic E. coli K-12 bacteria (available from LISBP),
Activated biotin (Perbio Science, EZ-Link Sulfo-NHS-LC-Biotin, ref. 21335),
Enzymatic probes of Neutravidin-HRP type (Perbio Science, ImmunoPure NeutrAvidin, HRP Conjugated, ref. 31 001),
Commercial, neutral PBS buffer (Perbio Science, BupH Phosphate Buffered Saline Packs, ref. 28 372),
Pyrogallol (Sigma Aldrich, ref. 25 4002),
Hydrogen peroxide (Roth, Hydrogen Peroxide 30% stabilised, ref. 8070),
Salts required for producing buffers and electrolytes:
   NaCl (Roth, ref. 3957),
   Na₂HPO₄, 2H₂O (Roth, ref. 4984),
   NaH₂PO₄, 2H₂O (Roth, ref. T879)

Material used:
Plate spectrophotometer (Multiscan Ascent),
Akta-Purifier liquid chromatography apparatus with UV characterizations (210, 254 and 280 nm) and collection system (GE Healthcare),
Size exclusion liquid chromatography column: Superose6 column (GE Healthcare, ref. 10/300 GL),
NanoSizer ZS particle sizer (Malvern Instruments).

Methods:
ATCC protocol for solid-phase phage amplification,
ISO 10 705-1 standard for phage counting in aqueous medium.

The first step for obtaining tracers entails the obtaining of suspensions of concentrated, purified, counted phages.

Phage Purification:

Concentrated phages can be obtained using known methods such as the ATCC protocol. The purification of suspensions of amplified phages is necessary to remove all bacterial debris and/or to release those phages still contained in host bacteria. Bacterial debris also contain lysine sites which may react with the molecules of activated biotin.

Two purification steps are needed. The first step is an extraction step with chloroform to lyse the remaining bacteria without damaging the bacteriophages (Adams, Bacteriophages, Intersciences Publishers, 1959). The chlororoform/suspensions assembly is centrifuged two times at 9000 rpm. The supernatant is collected then filtered through a sterile 0.2 micron filter. The suspensions are stored at 4° C. in their neutral PBS buffer and in glass containers to limit phenomena of protein adsorption. The storage conditions for the phages were chosen in accordance with data in the literature (Feng et al., Ind. Microbiol. Biotechnol., 30, 549-552, 2003) to minimize deterioration of the capsid over the longer term.

To quantify the size of the phages, particle size characterization was performed using a Nano ZS ZetaSizer (Malvern) (FIG. 2). The size distribution curve shows a single, reproducible peak centred on a mean value of 31 nm (value averaged over the five acquisitions). This mean diameter tallies fully with data in the literature.

Size exclusion liquid chromatography (HPLC-SEC) can also be used to quantify the phage concentration in the suspensions obtained (FIG. 3). The analyses are performed using a column having a molecular weight resolution range of between 5 and 5000 kDa (assuming a maximum load of 40 000 kDa). The molecular weight of native MS2 phages is 3600 kDa (Kuzmanovic et al., Structure. 2003 November; 11(11):1339-48.)

The eluent is neutral PBS buffer. The detections are made using UV spectrophotometry. The measured absorbency in this case at UV 254 nm is given as a function of the eluted volume. The smaller the eluted volume, the higher the molecular weight of the compound corresponding to this volume. In FIG. 3, the elution peak 1 centred on around 11.3 mL corresponds to the native phages. The peaks 2 correspond to proteins most probably derived from lysis of the bacteria or from the solid culture medium entrained when the amplified phages were placed in suspension. Since the surface areas of the peaks 1 are directly proportional to the concentrations of the compounds in the sample, the HPLC-SEC technique can therefore also be used for quantification of the phage suspensions.

The phage suspension could also be purified of the proteins by collecting the eluted fraction between 9.5 and 14 mL. However, purification is not carried through to completion so as not to dilute the phage suspensions.

Counting:

The precise determination of the concentration of purified phages allows determination of the tracer concentration. A count of the suspensions of purified phages can be carried out before the grafting step. The protocol followed is the one laid down by standard ISO 10 705-1.

The second step for obtaining the biotracer therefore consists of labelling the suspensions of phages obtained previously (i.e. the suspensions obtained after amplification of the phages, extraction with chloroform, centrifugation and 0.2 μm filtration) with the biotin molecules, and of removing the non-grafted biotin molecules by HPLC-SEC.

The activated biotin, added in very large excess, is directly dissolved in a few mL of phage suspension to promote contact between reagents. The reaction is conducted in a glass container at ambient temperature and neutral pH (i.e. the phage storage pH) and away from light. The mixture is agitated one and a quarter hours, then stored as such at least overnight at 4° C.

To remove the non-grafted biotin molecules, after the grafting reaction the mixture is purified by HPLC-SEC. The eluent is still neutral PBS. The chromatograms are given in FIG. 4.

This figure shows 3 curves:
curve 1: chromatogram of biotin alone, showing a characteristic peak of the molecule in the region of 20 mL,
curve 2: chromatogram of the phage suspension before grafting, showing the characteristic peak of the phages at around 11.3 mL and the residual proteins of the suspension on and after 20 mL,
curve 3: chromatogram of the suspension after grafting, showing a peak at around 11.3 mL corresponding to the grafted phages (the molecular weight of the biotin molecules is effectively too low for the variation in molecular weights [2.8%] between the native phages and grafted phages to be visible in the chromatograms) and a saturation peak beyond 20 mL corresponding to excess biotin.

The fraction corresponding to the phages labelled by the biotin molecules is collected between 9.5 and 14 mL in a glass tube and stored at 4° C. for subsequent labelling by the enzymatic probes. At this stage, it is not possible to say whether labelling by the biotin molecules has been effective. Only characterization of the mixture after reaction with the enzymatic probes will allow determination of the efficacy of this grafting.

The third step then consists of attaching the enzymatic probes on the biotinylated phages previously obtained, and of removing the non-attached enzymatic probes. The enzymatic probes are reconstituted in ultra-pure water then added in large excess to the fraction of biotin-labelled phages. The reaction is conducted at ambient temperature and neutral pH, away from light and under agitation (120 rpm) for 30 minutes.

To remove the non-grafted enzymatic probes, the post-reaction mixture is purified by HPLC-SEC. The molecular weight of the biotracers is estimated at more than 5000 kDa (taking into account the molecular weights of the enzymatic probes) but less than 40 000 kDa (maximum load of the chromatography column) which allows their recovery in the total exclusion peak. The eluent is still neutral PBS.

The chromatograms (FIG. 5) obtained at 210 nm, the wavelength at which the best response sensitivity of the enzymatic probes is obtained, show:
the enzymatic probe (curve 1) characterized by a peak centred on an approximate volume of 15 mL,
the suspension of biotinylated phages (curve 2) purified by HPLC-SEC, characterized as previously by a peak at around 11.3 mL,
the suspension of synthesized biotracers after grafting of enzymatic probes in large excess (curve 3): the peak of the biotracer can be seen centred at around 8.5 mL and the excess enzymatic probes (peak centred on 15 mL) (N.B.: a very slight offset can be seen between this latter peak and the peak in curve 1 since the batches of probes used in both cases did not have exactly the same characteristics: cf. supplier).

By comparing the curves 2 and 3, a distinct shift is ascertained in the peak of the biotinylated phages (11.3 mL) towards the smallest volumes (8.5 mL), with a significant deviation ΔV=2.8 mL. This translates an increase in the molecular weight of the native phages and guarantees the efficacy of biotin and enzymatic probe grafting, and hence the obtaining of the biotracers.

If the enzymatic probes are added in shortage, HPLC-SEC analysis allows visualization of the non-grafted biotinylated phages (curve 4, peak shoulder at 8.5 mL and peak at 11 mL).

The collecting of the biotracers is performed between 7.5 mL and 10 mL of elution volume (curve 3). The activity of the collected batches is characterized qualitatively by spectrophotometry by testing the chosen enzymatic activity with the adapted substrate. For example, HRP activity (oxidoreductase) was evaluated by reaction between pyrogallol and hydrogen peroxide, leading to a stained product in the presence of enzymatic activity. These positive qualitative tests rapidly confirm the presence and the activity of the enzymes, and indicate good biotracer production.

The biotracers are also characterized quantitatively to determine the quantity of grafted probes and to measure the catalytic activity of the biotracers. The purification of the biotracers by HPLC-SEC effectively allows collection of the fraction corresponding to the probe excess. This fraction is then assayed by spectrophotometry using a technique well known to a skilled person. The mass of the grafted probes is then deduced from the difference between the known mass of probes injected into the chromatography column and the collected quantity of excess probes. Knowledge of the mass of grafted enzymatic probes therefore allows the evaluation of a mean number of grafted probes per phage. It is then possible, if necessary, to express the quantity of tracers in grafted enzymatic probe equivalent, which allows measurement by spectrometry of the catalytic activity of the grafted probes (in other words the catalytic activity of the biotracers) using a technique well known to those skilled in the art. The same study can also be conducted using the amperometric method according to Example 2. It will be noted that successive purifications of batches of biotracers were conducted to ensure the efficacy of the purification method.

An example of the quantification of the grafted probes will now be detailed. The tracers are obtained in particular by labelling a suspension of purified biotinylated phages with enzymatic probes in excess at a concentration Cᵢ of 71.46±1.79 μg mL⁻¹ (following the previously described protocol), which therefore gives a mixture of tracers and of enzymatic probes in excess. Two peaks can be seen after HPLC-SEC purification of the mixture of tracers and excess probes (FIG. 8): the peak of the tracers (total exclusion peak) denoted T1 and the peak of free probes in excess (centred on 15.09±0.07 mL) denoted S1. In the remainder hereof, the probes in excess shall be called free probes in excess, in opposition to the probes attached to the phages relating to the tracers. The determination of the mass of probes grafted on the phages is therefore conducted by spectophotometric assay of the mass of free probes in excess and subtracting this mass from the injected mass of probes into the column (i.e. initially 71.46±2.00 μg).

Although the separation of the tracer peaks and the peaks of the free probes in excess is effective (with a mean separation coefficient <R>=1.29±0.1%), and although the concentration of free probes used for labelling (i.e. 71.46±1.79 μg mL⁻¹) was optimized (for complete grafting reaction and minimization of probe excess) the two peaks nevertheless show a less resolved band (between 12.00 and 13.50 mL elution volume) where the peaks do not return to the base-line (FIG. 8). Therefore only one part of each peak (V_{collT}1 and V_{collS}1 respectively) is collected and assayed; in particular, the collected volumes V_{collT}1 (between 7.50 and 10.00 mL elution volume) and V_{collS}1 (between 13.50 and 18.00 mL elution volume) are defined such that the risk of soiling is minimal between the collected fraction of the tracer peak T1 and the collected fraction of the peak of free probes in excess S1.

FIG. 9 illustrates details of the methodology used to access the mass mT1ₜₒₜₐₗ of probes attached to the phages. In FIG. 9, the curves 1 and 3 are those of FIG. 5, respectively relating to the enzymatic probe alone and to a mixture of tracers and excess probes. The following denotations are used:
C, the concentration of free probes used for labelling the phages,
V_{injection} the volume of the mixture of tracers and excess probes injected into the column,
mS0 the mass of probes injected into the column,
[S1]_{collected} the concentration of probes in the collected fraction of free probes in excess S1,
mS1_{collected} the collected mass of free probes in excess,
mS1ₜₒₜₐₗ the total mass of free probes in excess,
mS1T1 the mass of free probes in excess present in the collected fraction of tracers T1,
rS1 the ratio of the collected area under the peak of probes in excess S1 to the total area under peak S1,
mT1ₜₒₜₐₗ the mass of probes grafted onto the phages.

Initially, the concentration [S1]_{collected} of free probes in excess is determined in the collected fraction of free probes in excess S1 (between 13.50 and 18.00 mL) after purification, by spectrophotometric assay with pyrogallol following a technique well known to those skilled in the art. For this purpose, the specific enzymatic activity kcat_{PROBE}^{HPLC} of the free probes used for producing the tracers was previously measured, after passing through the HPLC column, by pyrogallol spectrophotometry: the determination of the concentration of free probes in excess in the fraction of collected free probes in excess S1 is therefore conducted using kcat_{PROBE}^{HPLC} (in the example given here, kcat_{PROBE}^{HPLC}=4.76 10⁴ min⁻¹, ±2.1%). The collected volume V_{colls1} of the fraction of free probes in excess S1 being known, it is therefore possible to access the collected mass mS1_{collected} of free probes in excess.

mS1_{collected}=[S1]_{collected}·V_{collS}1 (5)

Next, using the property of proportionality of HLC-SEC peak areas with masses, the parameter rS1 is used to access the total mass mS1ₜₒₜₐₗ of free probes in excess.

mS1ₜₒₜₐₗ=mS1_{collected}/rS1 (6)

At step 2, the total mass mT1ₜₒₜₐₗ of grafted probes is calculated by subtracting the total mass mS1ₜₒₜₐₗ free probes in excess from the mass mS0 of probes initially injected.

mT1ₜₒₜₐₗ=mS0−mS1ₜₒₜₐₗ (7)

The values of known parameters or parameters measured prior to determination of the total mass mT1ₜₒₜₐₗ of grafted probes in this example, are summarized in Table 1 below.

TABLE 1
Parameters known or measured prior to determination of the mass
mT1ₜₒₜₐₗ in this example.

| Known parameters | | |
|---|---|---|
| Cᵢ (μg mL⁻1) | 71.46 ± 1.79 | |
| V_{injection} (mL) | 1.00 | (±0.3%) |
| mS0 (μg) | 71.46 ± 2.00 | |
| V_{collS1} (mL) | 4.50 | (±0.6%) |
| V_{collT1} (mL) | 2.50 | (±0.6%) |
| Measured parameters | | |
| kcat_{PROBE}^{HPLC} (min⁻¹) | 4.76 10⁴ | (±2.1%) |
| rS1 (—) | 0.64 | (±2.2%) |

Table 2 below gives the total masses mT1ₜₒₜₐₗ of grafted probes and the intermediate masses used for calculation thereof, for three batches of tracers produced from the same batch of native phages, under the same synthesis conditions (following the previously described protocol).

TABLE 2
Total masses mT1ₜₒₜₐₗ of grafted probes for three batches of tracers
produced from the same batch of native phages, under the same synthesis
conditions.

| | Batch 1 | Batch 2 | Batch 3 |
|---|---|---|---|
| mS0 | 71.46 ± 2.00 | 71.46 ± 2.00 | 71.46 ± 2.00 |
| mS1_{collected} (μg) | 20.88 ± 0.79 | 21.20 ± 0.63 | 20.52 ± 0.75 |
| mS1ₜₒₜₐₗ (μg) | 32.66 ± 1.91 | 33.15 ± 1.68 | 32.09 ± 1.85 |
| mT1ₜₒₜₐₗ (μg) | 38.80 ± 3.91 | 38.31 ± 3.68 | 39.37 ± 3.85 |

To conclude, the approach taken provided access to the total mean mass <mT1ₜₒₜₐₗ> of grafted probes (i.e. 38.83±3.92 μg), which represents 54.3% of the total mass mS0 of probes initially injected into the column (i.e. 71.46±2.00 μg). This result translates the efficacy of the labelling and confirms the fact that by initially injecting a twice less quantity of probes (i.e. 35.30±0.82 μg), not all the biotinylated phages are labelled by the probes which are then in shortage (FIG. 5, curve 4). In addition, the results of Table 2 provide a quantitative indication on the good reproducibility of the protocol for tracer synthesis.

At this stage, it is then possible to determine the mean number of attached probes per phage. According to the supplier's data, the molecular weight M_{probe} of the enzymatic probes used for synthesis of the tracers is 140 kDa. Therefore the total mean mass <mT1ₜₒₜₐₗ> of grafted probes corresponds to a mean total number N of molecules of grafted probes: N=1.67 10¹⁴ probe molecules (±14.3%). Also, the mean quantity Q of tracers in the mixtures of tracers and free probes in excess to be purified, expressed in pfu equivalent, can be quantified from the mean concentration of biotinylated phages used for labelling the probes (i.e. 2.75±0.27 10¹² pfu mL⁻¹ deduced from measurement of the corresponding HPLC-SEC peak areas) and from the injection volume V_{injection} (having regard to the fact that the dilution used for labelling of the probes is negligible). Therefore, the mean quantity Q of tracers injected into the column is: Q=2.75 10¹² pfu (CV=10.1%). The mean number Nb of probes attached per phage is therefore deduced by dividing N by Q, i.e. Nb=61±18; which corresponds to nearly one third of the 180 available attachment sites on the capsid of the phages. In particular, a similar study conducted on a smaller suspension of native phages showed improved labelling efficacy.

The reproducibility of the labelling method set forth above was evaluated on nine batches of biotracers produced from the same batch of native phages under the same conditions. The results obtained (a qualitative study replicated 4 times and 2 quantitative studies: one replicated 3 times and the other 2 times) point to the very good reproducibility of the described method.

Particle size characterization of the biotracers can be performed. For example, the biotracers synthesized from MS2 phages labelled with biotin and the neutravidin-HRP complex show a mean diameter of 64.5 nm, making them fully pertinent for characterization of an ultrafiltration or microfiltration system.

This labelling technique can therefore be generalized to any protein capsid whose structures are compatible with labelling.

### Example 2

### Amperometric Detection

Step One:

In solution, if the enzyme functions at saturation (i.e. with no substrate-related limitation) and if the catalyzed reaction is irreversible, the following can be written:

[O](t)=[enzyme]ₜₒₜₐₗ·k_{cat}·t [equation 1]

where:

[O](t): concentration of oxidized donor formed in solution at time t (in mol·L⁻¹),

[enzyme]ₜₒₜₐₗ: total concentration of grafted enzymes present in solution (in mol·L⁻¹),

k_{cat}: molar activity of the grafted enzyme (in mol·mol⁻¹·min⁻¹ or min⁻¹),

t: time of the reaction catalyzed by the grafted enzymes (min).

The constant k_{cat} more explicitly translates the number of molecules of oxidized donor formed in solution per enzyme molecule present in solution and per unit of time.

For the chosen enzyme, whose kinetics can be modelled by kinetics of Michaelis-Menten type (given in the literature: Veitch et al. Phytochemistry 65, 249-259 (2004), experimentally verified), the enzyme functions at saturation when the concentration of substrate [S] is not limiting: [S]>10×Km (Km=Michaelis-Menten constant in mol·L⁻¹ of the chosen enzyme). For the purposes of this study the fixed concentration [S] is always chosen so as to meet this condition, irrespective of the range of enzyme concentrations used.

Step Two:

For a schedule controlled by diffusion, the current generated at time t:l(t), related to the reduction of the oxidized donor on the electrode, is the limiting diffusion current. This current is a cathodic reduction current, hence counted negatively as per the following law:

l(t)=−n·F·S·Do·[O](t)/δ [equation 2]

where:

l(t): reduction current generated at time t (A or C·s⁻¹),

[O](t): concentration of oxidized donor formed in solution at time t (mol·m⁻³),

n: nb of e− exchanged (−),

F: Faraday constant (C·mol⁻¹),

S: active electrode surface (m²),

Do: coefficient of diffusion of O towards the RDE (m²·s⁻¹),

δ: diffusion layer (m).

This law translates the decrease in current with the increase in quantity of oxidized donor formed over time.

The transfer of the oxidized donor to the working electrode is controlled by diffusion if the hydrodynamics in the solution are fixed by the working electrode: in this case a rotating disk electrode, and if the speed of rotation of the electrode ω (rpm⁻¹) is chosen so that at any one time the quantity of oxidized donor O consumed at the electrode is less than 1% of the total quantity of oxidized donor O present in solution.

By adding equation 1 to equation 2, this gives:

l(t)=−n·F·S·Do·k_{cat}·t·[enzyme]ₜₒₜₐₗ/δ [equation 3]

After a certain time, this current stabilizes (due to total oxidation of the electron donor R). Under these conditions, the concentration of oxidized donor O is constant and the limiting diffusion current is constant. It is called the limiting diffusion current in stationary mode and is denoted l_{stationary}.

According to equation 3, it therefore appears that if a sufficient time t is chosen for the current to be significant, it is possible to correlate the current measured at this time with the total quantity of enzymes present in solution.

However, for greater accuracy and to be free of any current reference constraint, preference can be given to using the slope through the origin Vo (A·s⁻¹) defined by deriving equation 3 in relation to time:

Vo=(dl/dt)ₜ₌₀=−n·F·S·Do·k_{cat}·[enzyme]ₜₒₜₐₗ/δ=constant [equation 4]

i.e. in absolute value: |Vo|=|(dl/dt)ₜ₌₀=n·F·S·Do·k_{cat}·[enzyme]ₜₒₜₐₗδ [equation 5]

Therefore, by experimentally measuring the slope Vo and with a calibration curve obtained by amperometry and/or by spectrophotometry, it is possible to access the quantity of grafted enzymes in the measuring cell and hence the quantity of biotracers in the measuring cell and, with volume considerations, the quantity of tracers in the sample.

To explain the combined use of the biotracer and the detection method in the study under consideration, a general flowchart is given in FIG. 6.

Said study conducted at different times provides knowledge on changes in biotracer concentrations in the permeate, during filtration. The feed and the retentate (or concentrate) can be analysed using the same approach.

### 1.1. Measuring System and Protocol

a. Reagents and Materials

Reagents:
3,3′,5,5′-Tetramethylbenzidine also called TMB (SIGMA Aldrich, ref. 87750),
Hydrogen peroxide (ROTH, Hydrogen Peroxide 30% stabilised, ref. 8070),
Salts required for producing buffers and electrolytes:
   NaCl (Roth, ref. 3957),
   Na₂HPO₄, 2H₂O (Roth, ref. 4984),
   NaH₂PO₄, 2H₂O (Roth, ref. T879),
   Citric acid (Sigma, ref. 251275).

Material:

A complete amperometric detection system (Metrohm).

The chosen potentiostat is MicroAutolab (Metrohm), having a resolution of 30 fA and measuring nA in repeatable fashion. The amperometric cell is a thermostatable, Karl Fisher glass cell (Metrohm) with flat lid to allow easier cleaning and positioning of the electrodes, of maximum volume 130 mL. Two cannulas for dinotrogen bubbling can also be positioned on the lid. The working electrode chosen is a Metrohm rotating disk electrode (RDE) whose active surface is a platinum disk 5 or 3 mm in diameter adapted to the work volume of the cell. The working electrode has a mercury contact between the rotating shaft and the body of the electrode, thereby ensuring signal transmission with noise minimization. The servo-control system for the RDE permits a rotation speed of between 100 and 10000 rpm. The counter-electrode is a platinum wire electrode with a chosen active surface 6.5 times larger than that of the working electrode. The chosen reference electrode is a double-junction Ag/AgCl electrode.
It will be noted that the parameters concerning cell volume, working electrode material and surface, counter-electrode surface and geometry may vary in relation to the optimization of the detection system and the different desired applications.

b. Measurement Protocol

The chosen enzyme allows numerous possible electron donors. For the application and in the light of the literature (Volpe et al., Analyst, June 1998, Vol. 123 (1303-1307)), the electron donor used for this example is TMB. Since the enzyme is HRP, the oxidant is hydrogen peroxide. The ratio of the concentration of hydrogen peroxide to the concentration of TMB is set at 5.

The maximum total volume of solution to be analyzed is fixed at 78 mL for this system. The concentrations of reagents: i.e. TMB and hydrogen peroxide are chosen so that the enzyme functions at saturation. The quantity of TMB is smaller than the quantity of hydrogen peroxide to limit spontaneous reaction. The chosen buffer electrolyte is a citrate-phosphate buffer pH=5 at 0.1 mol·L⁻¹ also containing 0.1 mol·L⁻¹ of NaCl electrolyte. A 5 mm platinum disk is chosen as active surface of the working electrode. The rotation speed of the RDE is set at 1130 rpm.

Before taking any measurement, analysis of the blank (i.e. in the presence of TMB and hydrogen peroxide only) is conducted to quantify the share of spontaneous reaction on the current produced and hence on the measured slopes through the origin.

The mechanism of TMB oxidation is a two-step mechanism (Josephy et al., The Journal of Biological Chemistry, Vol. 257, no 7, Issue of April 10, pp. 36669-3675, 1982) generating two oxidized forms of TMB: an intermediate, partly oxidized compound denoted TMBox1 at step one, and a fully oxidized compound denoted TMBox2 at step two.

Either TMBox1 or TMBox2 can be chosen for assay. If TMBox2 is assayed (Fanjul-Bolado et al., Anal. Bioanal. Chem. (2005) 382: 297-302), the measurement protocol comprises:
a step for incubating the solution containing at least one biotracer, the reagents TMB and hydrogen peroxide, and the buffer electrolyte. This incubation step is performed in the amperometric measuring cell for 1 to 30 min under agitation,
a step for adding concentrated phosphoric acid so as to oxidize all the TMBox1 which may be present in solution in TMBox2,
a step for measuring current the minute following after the adding of phosphoric acid.

In this example, TMBox1 is preferably assayed. To analyse a sample, it is placed in the measuring cell. The polarisation voltage is fixed at 240 mV. The maximum volume which can be sampled is set at 60 mL. The volume of the cell, from which the volume of reagents is deducted, is completed with the buffer electrolyte. The RDE is set in rotation, this being sufficient to ensure mixing of the volume to be analyzed. The TMB is then added followed by hydrogen peroxide. As soon as the hydrogen peroxide is added, acquisition is initiated. Analysis is conducted at ambient temperature without deoxygenation of the solution. The acquisition time for the blank is fixed at the maximum duration of analysis. The acquisition time for analysis of the samples is chosen so that the number of acquired points is sufficient to obtain good accuracy of measurement of the slope Vo. The duration of analysis varies from 1 to 15 min.

FIG. 7a shows an example of amperometric curves. Four volumes of one same biotracer feed were analyzed, corresponding to four concentrations of biotracers in the measuring cell, expressed in native phage count units: C0, 2.00×C0, 4.65×C0 and 5.80×C0. FIG. 7b shows the calibration line associated with the example (giving the slopes measured as a function of tracer concentrations in the cell). The linearity of the slope with tracer concentration translates the fact that, over this range of concentrations, grafting can be considered to be uniform.

FIG. 10 gives an example of amperometric responses obtained by analyzing the feed and the permeates collected at the end of filtration, for a microfiltration membrane MF and an ultrafiltration membrane UF. In particular, the response obtained for the MF permeate evidences that the microfiltration membrane partly allows the passing of tracers, since the amperometric slope corresponding to the MF permeate is less steep than the feed slope. The response for the UF permeate is similar to that of the blank, which means that no tracers were detected in the UF permeate. The results given in FIG. 10 therefore highlight the capability of the developed method to characterize different membrane behaviours.

FIG. 11 gives an example of dynamic characterization of the retention obtained with an ultrafiltration module having damaged hollow fibres used for the production of potable water. During this experiment, tracer increments were applied in particular for around one third of the total filtration time (conducted frontally at constant transmembrane pressure) and the permeate was collected during filtration for analysis. The tracer concentrations measured in the permeate collected during filtration are given as a function of filtration time. The results obtained particularly show that dynamic monitoring of tracer retention by the membrane is possible. It was therefore able to be observed that the tracer concentration increased initially to reach a maximum towards the mid-injection time and then decreased.

1-25. (canceled)

26. Method for detecting by amperometry, in a sample to be analysed, a biotracer consisting of a labelled bacteriophage comprising on its surface one or more enzymatic probes grafted via one or more molecules of activated biotin previously attached to one or more proteins of the capsid of said bacteriophage.

27. The method according to claim 26, such that said detection method comprises:
preparing an amperometric cell comprising:
a working electrode,
a counter-electrode,
a reference electrode,
these three being connected by a potentiostat-galvanostat;
a solution comprising the sample to be analysed and at least one biotracer as defined above,
an electrolyte,
an oxidant,
an electron donor (R),
and
measuring by amperometry the current thus generated.

28. The method according to claim 27, wherein the electron donor is chosen from the group composed of iodide or 3,3′,5,5′-tetramethylbenzidine.

29. The method according to claim 27, wherein the working electrode consists of a material chosen from the group consisting of platinum, glassy carbon or gold.

30. The method according to claim 27, wherein the counter-electrode is a platinum electrode.

31. The method according to claim 27, wherein the oxidant is hydrogen peroxide.

32. The method according to claim 26 such that the bacteriophage is a MS2 phage.

33. The method according to claim 27, such that the electrolyte further comprises a buffer.

34. The method according to claim 26, such that said molecules of activated biotin are chosen from among biotins capable of reacting with primary amine groups.

35. The method according to claim 26 such that said enzymatic probe is a complex containing:
a protein carrier capable of interacting with the activated biotin, and
an enzyme of oxidoreductase type.

36. The method according to claim 35, wherein said protein carrier is chosen from among neutravidin, avidin or streptavidin.

37. The method according to claim 35, such that said enzyme is Horse Radish Peroxidase (HRP).

38. Method for monitoring a filtration system, comprising:
adding one or more biotracers consisting of a labelled bacteriophage comprising on its surface one or more enzymatic probes grafted via one or more molecules of activated biotin previously attached to one or more proteins of the capsid of said bacteriophage, to the feed of said system,
the step of detecting said biotracer(s) in the permeate and/or in the retentate of said filtration system by amperometry, according to claim 26.

39. Method according to claim 38, further comprising:
the step of detecting biotracers in the feed of said system, then
comparing the current obtained in the permeate and/or retentate with the current obtained in the feed.

40. A biotracer consisting of a labelled MS2 bacteriophage, said bacteriophage comprising on its surface one or more enzymatic probes grafted via one or more molecules of activated biotin previously attached to one or more proteins of the capsid of said bacteriophage.

41. The biotracer according to claim 40, such that the enzymatic probe(s) and/or the molecule(s) of activated biotin are such as previously defined.

42. A method for preparing the biotracer according to claim 40, comprising the following steps:
immobilizing one or more molecules of activated biotin on at least one protein present on the surface of the bacteriophage to be labelled, then
grafting an enzymatic probe on said biotin(s).

43. The method according to claim 42, further comprising the step of purifying the labelled biotracer thus obtained.

44. The method according to claim 43, such that said purification is performed by HPLC-SEC.

45. The method according to claim 42, further comprising a preliminary step of producing bacteriophages to be labelled by amplification, followed by placing in suspension and purification.

46. The method according to claim 42, wherein said molecule of activated biotin is immobilized on a lysine of the surface proteins of the bacteriophage.

47. The method according to claim 42 further comprising the step of quantifying the mean number of grafted probes per bacteriophage.

48. A kit comprising:
a solution containing at least one biotracer consisting of a labelled bacteriophage comprising on its surface one or more enzymatic probes grafted via one or more molecules of activated biotin previously attached to one or more proteins of the capsid of said bacteriophage, and
an amperometric cell comprising a working electrode, a counter-electrode, a reference electrode,
an electrolyte,
an electron donor, and
an oxidant.

49. The kit according to claim 48 further comprising a buffer.

50. The kit according to claim 48 such that said solution, the said electrodes and/or biotracer are as previously defined.
